(19) United States
(12) Patent Application Publication
(10) Pub. No.: US 2015/0122269 A1
(43) Pub. Date: May 7, 2015

(54) TOPSHEET CONTAINING JADE PARTICLES AND METHOD FOR ITS MANUFACTURE

(71) Applicant: James W. CREE, Loveland, OH (US)

(72) Inventor: James W. CREE, Loveland, OH (US)

(21) Appl. No.: 14/526,818

(22) Filed: Oct. 29, 2014

Related U.S. Application Data
(60) Provisional application No. 61/898,829, filed on Nov. 1, 2013.

(51) Int. Cl. A61F 13/513; A61F 13/512; A61F 13/15; A62B 23/02; A61F 13/49; A61F 13/472; A41D 13/11; A61F 13/511; A61F 13/539

(52) U.S. Cl. CPC A61F 13/513; A61F 13/51121; A61F 13/5126; A61F 13/15642; A61F 13/539; A61F 13/49; A61F 13/472; A41D 13/11; A62B 23/025; A61F 2013/5105
USPC 128/863; 604/367; 604/366; 427/2.3

(57) ABSTRACT

A topsheet made of a fibrous web or a perforated film and having a clear top layer containing about 5% to about 50% of jade particles. The jade particles having a mean diameter of from about 50 to about 600 microns and an aspect ratio from about 1 to about 5. Also disclosed is method for manufacturing the topsheet, as well as absorbent articles and facial protection masks containing the topsheet.

### CROSS-REFERENCE TO RELATED APPLICATION

This application claims the benefit of co-pending U.S. Provisional Application No. 61/898,829, filed Nov. 1, 2013.

### FIELD OF INVENTION

The present invention relates to topsheets containing jade particles and methods for manufacturing them. The topsheets may be perforated films or fibrous webs, which may be woven or nonwoven. The topsheets are useful for absorbent articles and as filtration inserts for facial protection masks.

### BACKGROUND OF THE INVENTION

Disposable absorbent articles known in the art include diapers, bandages, catamenials, and the like. Such articles generally comprise a fluid permeable topsheet, a fluid impervious backsheet, and an absorbent element between the two. The topsheet overlays the absorbent element and forms the surface that contacts the wearer. A topsheet should rapidly absorb liquid and pass it on into the absorbent element, prevent absorbed liquid from re-wetting the surface, provide a dry surface having a pleasant, textile like feeling on the skin, be pliable and soft, securely encase the absorbent element, and be thin, easy to manufacture and cost effective.

Topsheets known in the art include fibrous webs, which may be woven or non-woven (carded or spunbond), and perforated plastic films. While fibrous webs may be preferred from a standpoint of haptics, topsheets made from perforated plastic films may have advantages in being easy to produce and cost effective. The form of the perforations allows one to regulate properties such as fastness of absorption into the absorbent element and re-wet upon applying pressure. On the other hand, plastic films may lack the soft, textile like touch of woven and non-woven webs, and may have a sticky surface. Numerous approaches have been tried to improve the touch of topsheets from plastic films.

The most common approach is to texture the surface of the film or impart a three-dimensional pattern by embossing. A textured surface may be combined with a three-dimensional structure made by embossing. The structures may range from very small, microscopic structures to larger structures perceivable with the naked eye.

EP 018 684 discloses providing microscopic nubbles on the surface of the film. The nubbles are made by an embossing step using a roller to which particles with a mean diameter of 0.04 to 0.08 mm embedded in an epoxy resin are applied.

U.S. Pat. No. 6,548,158 discloses using a multilayer film with an outer layer containing 30 to 60 parts by weight of a filler with a particle size from 2 to 20 microns to roughen the surface.

EP 1 946 734 A1 discloses perforated multilayer films with a top layer comprising a thermoplastic material containing 5 to 25% by weight of inert particles with a mean diameter of from about 1 to 5 microns. The invention is said to provide soft, cloth like topsheets that have fast absorption, a dry surface, and mechanical stability.

Jade is an ornamental stone that has long been revered as having protective and healing properties, particularly in Asian cultures. Jade stones have been shaped and added to many articles.

### SUMMARY OF THE INVENTION

In one embodiment, the invention relates to a topsheet comprising a fibrous web or a perforated film, said topsheet having a clear top layer comprising from about 5% to about 50% by weight of jade particles having a mean diameter of from about 10 to about 600 microns and an aspect ratio from about 1 to about 5.

In another embodiment, the invention relates to a method for manufacturing a topsheet for an absorbent article or facial protection mask, comprising the step of forming a clear top layer comprising from about 5% to about 50% by weight of jade particles having a mean diameter of from about 10 to about 600 microns and an aspect ratio from about 1 to about 5 on a fibrous web or film.

In another embodiment, the invention relates to an absorbent article or facial protection mask comprising a topsheet comprising a fibrous web or a perforated film, said topsheet having a clear top layer comprising from about 5% to about 50% by weight of jade particles having a mean diameter of from about 10 to about 600 microns and an aspect ratio from about 1 to about 5.

### DETAILED DESCRIPTION OF THE INVENTION

The topsheets of the invention comprise a fibrous web or a perforated film having a clear top layer comprising from about 5% to about 50% by weight of jade particles. Since the top layer is clear, at least some of the jade particles are visible to provide the aesthetics desired by the user, including the perceived protective and healing properties when the topsheet is used in an absorbent article or a facial protection mask.

The jade particles may comprise any kind of jade and other material acceptable for use in articles contacting the human skin and having the desired mean diameter. The particles may be coated with various materials, such as with fatty acid esters. The mean particle size should range from about 10 microns to about 600 microns, typically from about 50 microns to about 400 microns, and more typically from about 100 microns to about 300 microns. The particle size is determined by sieving with sieves of different sizes. The mean particle size is characterized by the fact that 50% of all particles have a size equal or smaller than that value. The particles preferably are at least about 100 microns long in at least one dimension to be visible to the naked eye, but less than about 600 microns long in every direction so as to not be so large that they may feel scratchy to the user of the topsheet or article containing the particles.

The aspect ratio (length/width-ratio in all three dimensions) of the jade particles typically ranges from 1 to 5, typically from 1 to 3, more typically from 1 to 2. Preferred are particles having an aspect ratio close to 1, i.e., more or less spherical particles. The aspect ratio is determined by counting several (at least 10) particles photographed via REM-microscope and measuring their greatest length and smallest width. The aspect ratio is the quotient of length and width.

The clear top layer of the topsheet comprises at least about 5% by weight of the jade particles, typically more than about 10% by weight. The top layer comprises less than about 50% by weight, typically less than about 30% by weight, of jade particles to avoid having too high a stiffness and hardness. The top layer typically comprises from about 10% to about 25% by weight, e.g., from about 10% to about 20% by weight, of jade particles. At least some of the jade particles in the top layer are visible to provide the aesthetics desired by the user.

In one embodiment, the topsheets of the invention comprise a perforated film. The top layer of the film comprises one or more thermoplastic materials like polyolefins. The thermoplastic material typically is a polyethylene, particularly a low density polyethylene (LDPE) or a linear low density polyethylene (LLDPE). The LLDPE may be one made with conventional catalysts like Ziegler-Natta catalysts or it may be made by Metallocene catalysts (m-LLDPE). It also possible to use a mixture of two or more of the said materials, for example, a mixture of LDPE and LLDPE, or a mixture of LDPE, LLDPE and m-LLDPE, or a mixture of LLDPE and m-LLDPE.

The top layer may contain additional ingredients like additives in their usual amounts provided the top layer remains at least substantially clear so that the jade particles are visible to the user. The top layer may thus comprise one or more of processing aids, antistatic agents, antislip agents, stabilizers, antioxidants, acid neutralizing agents, ultraviolet absorbers, antiblocking agents and antifogging agents. The additives are used in their usual amounts, typically up to 5% additives,

The top layer may comprise at least one additional layer apart from the top layer. It may comprise one, two, three, four, five or even more additional layers. Typically, the top layer may comprise one or two additional layers, usually just one additional layer.

The composition of the additional layer is not specifically restricted. Typically, the additional layer is coextrudable or, less commonly, bonded via direct extrusion of one of the layers onto the other. Thus the materials should be able to provide suitable adhesion with the top layer upon coextrusion or direct extrusion. In the case of two or more additional layers, they can comprise the same or different components. Usually they will differ in composition. It is also possible to laminate a top layer with the additional layer(s) by any means known in the art, such as thermobonding or adhesives.

Suitable materials for the additional layer(s) are thermoplastic materials like LDPE, LLDPE, mLLDPE, and PP-homopolymers or copolymers. Typically, the materials are the thermoplastic materials described above for the top layer.

The additional layer(s) may comprise usual ingredients like fillers, additives, colorants and so on in their usual amounts. Thus they may additionally comprise common additives like colorants, mineral fillers, processing aids, antistatic agents, antislip agents, stabilizers, antioxidants, acid neutralizing agents, ultraviolet absorbers, antiblocking agents and antifogging agents. The additives are used in their usual amounts, typically up to 5% additives, with colorants up to 15% by weight. The additives in the additional layers may be the same or different and be present in the same or differing amounts in case more than one layer is present.

In one embodiment, the additives may include mineral fillers, such as calcium carbonate, glass beads and barium sulfate, and especially calcium carbonate. The filler particles may be coated in a manner known as such, e.g. with fatty acid esters. The mean particle size should range from about 1 micron to about 5 microns, typically from 2 microns to 4 microns, and more typically from 2.5 microns to 3.5 microns. The particle size is determined by sieving with sieves of different sizes. The mean particle size is characterized by the fact that 50% of all particles have a size equal or smaller than that value. The size of the particles typically ranges from 0.1 to 15 microns, e.g., from 1 to 10 microns.

In one embodiment, the film (prior to perforation) has two layers with the top layer adjacent to the skin containing more than 10% by weight of jade particles, e.g., 15%, and 50 to 80% by weight of m-LLDPE, e.g., 75%, and additives like antifogging agents and processing aids in an amount of 10%. The additional layer contains 50 to 90% by weight of LDPE, e.g., 75%, 10 to 20% by weight of LLDPE, e.g., 15%, and additives like colorants, processing aids and antifogging agent up to 10% by weight of the layer. After perforation, the thickness of the top layer is between 50 and 200 microns, and the thickness of the additional (inner) layer is between 200 and 600 microns.

In a second embodiment, the film is a film has two layers with the top layer adjacent to the skin containing more than 10% by weight of jade particles, e.g., 15%, and 50 to 80% by weight of LDPE, e.g., 75%, and additives like antifogging agents and processing aids in an amount of 10%. The additional layer contains 50 to 90% by weight of LDPE, e.g., 70%, 10 to 30% by weight of LLDPE, e.g., 20%, and additives like colorants, processing aids and antifogging agent up to 10% by weight of the layer. In one example, after perforation, the thickness of the top layer is 10 microns and the thickness of the inner layer is 450 microns.

Suitable LDPE is commercially available and has a MFI (190 C/2.16 kg) from 1.95 to 2.5 g/10 min, preferably 2 g/10 min and a melting point from 110 C to 120 C, preferably 116 C. Suitable m-LLDPE is commercially available and has a MFI (190 C/2.16 kg) from 2.5 to 3.5 g/10 min, preferably 3 g/10 min and a melting point of from 60 C to 80 C, preferably 68 C. Suitable LLDPE is commercially available and has a MFI (190 C/2.16 kg) from 4 to 5 g/10 min, preferably 4.4 g/10 min and a melting point from 110 C to 130 C, preferably 127 C.

The thickness ratio of the top layer to the additional layer(s) is typically from 1:2 to 1:50, especially from 1:3 to 1:10. The thickness of the films typically is in the range of 15 to 500 microns, more typically 20 to 200 microns.

The films may be manufactured and perforated according to any method known in the art, for example, as disclosed in EP 1 946 734 A1, incorporated herein by reference.

Typically a precursor film will be formed by blown film extrusion or cast extrusion of molten thermoplastic material containing the jade particles and any other desired material. At least some of the jade particles in the clear top layer of the film will be visible to the user. The precursor film may also be textured and/or embossed upon extrusion.

The precursor film is then perforated, typically by vacuum and application of hot air.

The film may further be stretched, ring rolled, printed, etc. after or before perforating. Stretching serves to reduce the thickness of the film, and stretching and ring-rolling improve the mechanical properties. Ring-rolling can also impart or improve elasticity of the film. The surface feeling of films can be improved, i.e. the haptics can be made more similar to that of textiles, by cold embossing a perforated and textured film.

Thus, the present invention also provides a method for manufacturing perforated films suitable as a topsheet comprising the steps of providing a precursor film, texturing the surface of the film, perforating the film and embossing the perforated film at a temperature from 15 to 70 C.

With the method of the invention, the surface of the films becomes soft and textile like, while the liquid absorption and re-wet properties remain excellent.

The first step of the method is manufacturing a precursor film whereby the surface of the film is textured, e.g., via common embossed cast film rollers.

The film may be a mono layer film from thermoplastic materials as described above for the top layer. Typically, the precursor film is a multilayer film as described above, i.e. with a top layer or top layers from a filled thermoplastic material as described above.

The texturing may be of any pattern, for example, diamond shaped cells. They may have a width and length of 200 microns each, and a depth of 45 microns, arranged in the machine direction.

The film is then perforated by means known in the art. It is also possible to store the film and perform the perforation at a later time (off-line).

Perforation is typically accomplished by passing the heated film over an apertured roll having a vacuum applied from the inside. The film may be heated by passing it over heated rollers, by infrared radiation and/or by directing a stream of hot air onto the film. The thermoplastic material softens and the vacuum causes the film to be sucked into the apertures and finally rupture so that perforations result. The process used is described in detail in U.S. Pat. No. 4,151,240. The device includes a constant tension film supply before the perforation drum and constant tension forwarding and winding. To perforate the film, a heating hot air jet is placed against one surface of the film while applying vacuum adjacent the opposite surface of the film. For example, the film may be heated by means of hot air (350-550 C) to a temperature near the melting point (for PE around 110 C and 130 C) and sucking the hot web on a porous roll.

The structure of the apertures in the roll determines the structure of the perforations. The vertical profile, form and size of the apertures has to be adapted to provide perforation of the intended shape as is per se known in the art.

The vertical profile of the perforations, i.e. the form as viewed perpendicular to the film surface, influences the absorption and re-wet properties. Perforations with a diameter that is smaller on the side of the absorbing pad than on the body facing side will considerably lessen re-wet. Thus, it is preferred to use apertures providing conical or other non-uniform diameter perforations with respect to the vertical profile.

The outer contour or form may be of any kind, for example, simple, convex polygons, like rhombus, trapeze, quadrate, rectangle, pentagon, hexagon, octagon and so on, and structures with curved contours, like circles and ellipses. It is also possible to use forms combining straight lines and curves like rectangles with circular ends.

The size of the perforations should range from 0.1 to 10 mm², typically from 0.2 to 7 mm² (0.5 to 3 mm diameter), e.g., around 3 mm². The mean distances between the perforations should be 0.1 to 1.0 mm, e.g., about 0.4 mm. The actual distance will vary for some patterns like ellipses or circles and vary for different directions with patterns like rhombus or hexagon. Typically one uses approximately equal distances between the perforations in all directions, i.e. to arrange them evenly.

The pattern and/or arrangement influences the mechanical properties whereas the size of pattern influences both mechanical and hygiene properties.

It is also possible to use other means of perforation, like needling or jets of liquid etc. known in the art.

The perforation step may also be preceded by other steps like printing, stretching and so on.

The perforated film is then embossed at a temperature of up to 70 C, typically up to 60 C, more typically up to 50 C. The lower limit of the temperature depends on the material. For useful thermoplastic materials, typically it is between 10 C and 20 C, usually around 15 C. It is important that this embossing is performed at comparably low temperatures. When the higher temperatures like 80 C or more are used, the soft, textile like touch is not achieved. Furthermore, especially for perforation patterns with large hole size, the strike-through properties will be reduced dramatically during hot embossing due to closing the pores by melting material.

Generally speaking, the bigger the perforation size, the lower embossing temperature is needed. With small perforations, a higher embossing temperature is possible. There will generally be an optimum temperature with a view to haptic properties that can be determined by tests for a given material, perforating and embossing pattern. This temperature will not always be optimum with respect to re-wet and strike through, i.e., hygienic properties. In such instances a compromise between optimizing haptics and hygienic properties may be found by way of tests.

The pattern of the embossing should differ from the texture applied directly after extruding the film. Otherwise, it is not specifically restricted. Patterns include lines, straight and curved, crossing lines, interrupted lines, dots, and points, e.g., rectangular or other straight lines crossing each other.

Typically, the lines have a distance from each other of 100 to 800 microns, more typically 200 to 600 microns, e.g., 300 to 500 microns.

Before and after the embossing station, there is usually a system to control the tension of the film. The film is driven between two rolls, one roll with a special embossing and the counter roll with a smooth surface, which can be, e.g., metal, rubber or paper. Both rolls turn in opposite direction. In one embodiment, the roll and the counter roll are manufactured from a rigid material and are mounted with an adjustable pressure between the two rolls. The embossed roll is temperature regulated and from metal.

As stated above, the film may additionally be printed, stretched, ring-rolled or subjected to any other known process, with the exception of embossing at high temperatures, calendaring and the like. In one embodiment the film is not stretched or ring-rolled.

The films are suitable for use as topsheets for absorbent articles like catamenial pads, diapers, incontinence products and the like. Thus, the invention also relates to such articles comprising the films as a topsheet, whereby the top layer of the films forms the body-facing side of the topsheet.

In another embodiment, the topsheets of the invention comprise a fibrous web, which may be any suitable woven or nonwoven (carded or spunbond) web. The fibrous web has a clear top layer comprising from about 5% to about 50% by weight of jade particles. The fibrous web typically is a nonwoven web, which may be formed by techniques known in the art and/or commercially obtained. The nonwoven web may be any porous web that has a fibrous appearance. Nonwoven webs can be made by various processes using carded fibers that are thermally bonded, airthrough bonded, or directly extruded via a process called spunbond or meltblown. The nonwoven web has a basis weight between about 9 gsm and about 80 gsm, typically between about 10 and about 30 gsm, e.g., between about 10 and about 16 gsm. The nonwoven fibers are predominantly polyethylene or polypropylene, or blends thereof. The nonwoven typically is hydrophilic, or it can be a layered airthrough nonwoven with the side facing the absorbent core being more hydrophilic than the side that contacts the microperforated coating. In one embodiment, the nonwoven has a capillary gradient such that capillaries closer to the absorbent core are smaller than ones adjacent the coating layer. In the absorbent artice case, the nonwoven layer facing the core may have higher affinity to fluid, which can be created by using a more permanent hydrophilic material that lowers the contact angle of the web or by using capillary channeled fibers.

The fibrous material may be intrinsically hydrophilic and/or may be rendered hydrophilic by techniques known in the art. A surface treatment having a relatively lower surface energy may be applied to the top layer of the web and cured. A suitable surface treatment is a UV-curable silicone such as disclosed in U.S. Pat. No. 6,025,535, incorporated herein by reference. The UV-curable silicone may be blended with a photocatalyst, in proportions by weight of 100 parts to 2.5 parts, respectively, and applied to the web at a rate of between about 1.5 and about 2.0 grams per square meter (gsm). For example, jade particles having a mean diameter ranging from 10 to 400 micron are blended into the liquid silicone at a loading range not to exceed 50 parts per 100 parts silicone. The slurry mix is then applied to the surface of the nonwoven and the web is cured. The jade particles are immobilized on the surface of the nonwoven after passing the web under a UV curing light. The silicone coating technique can also be used to apply jade particles to a perforated film or to a barrier film, eliminating the need to introduce the jade particles inside the thermoplastic resin as a second phase particle.

Surface treatments containing the jade particles and materials such as the above-described UV-curable silicone may be applied to the top layer of the fibrous web by techniques known in the art, such as screen printing, gravure printing, spraying, dip coating, etc. One approach for applying surface treatments to a continuous web include application of the treatment via a smooth roll printing apparatus such that the resulting regions exhibit a random, non-visually-discernible pattern (i.e., a pattern which is not visible to the normal naked eye when viewed at a distance of approximately 12 inches in a direction normal to the web surface). In addition, the regions may be of a sufficiently fine scale that they may not be individually identifiable at a like distance such that the regions impart an overall visual property to the web in the aggregate rather than by providing a visually-discernible alternating pattern. At least some of the jade particles in the clear top layer will be visible to the user.

When such a silicone blend is utilized on a nonwoven web, the coating application levels may be about 1.5 to about 2.0 grams silicone per square meter of web surface area, although other coating levels may prove suitable for certain applications depending upon the nature of the web material and surface, the characteristics of the fluid, etc.

Other suitable treatment materials include, but are not limited to, fluorinated materials such as fluoropolymers (e.g., polytetrafluoroethylene) and chlorofluoropolymers. Other materials which may prove suitable for providing regions of reduced surface energy include Petrolatum, latexes, paraffins, and the like, although silicone materials are advantageous for use in fluid-previous webs in the absorbent article context for their biocompatibility properties.

The invention will be illustrated by way of the following examples, which shall not be construed to limit the scope of the invention to the specific embodiments described. If not stated otherwise, all percentages and parts are given by weight.

### EXAMPLE 1

### Silicone with Jade Applied to a Nonwoven Topsheet

UV curable silicone is blended with a photocatalyst. Jade particles having a mean diameter of about 100 or 200 microns and an aspect ratio about 1 or 2 are included in the mixture and stirred to distribute evenly. The silicon mixture is then applied to the surface of the nonwoven web using a kiss roll transfer technique. The silicone coated nonwoven is then exposed to a UV lamp and the silicone with the jade particles embedded therein is cured and remains on the surface of the nonwoven web.

### EXAMPLE 2

### Jade Embedded in the Extrusion of a Perforated Film

Jade particles having a mean diameter of about 100 or 200 microns and an aspect ratio about 1 or 2 are introduced along with LDPE and LLDPE resin into one of the two hoppers of a coex cast extruder. The hopper feeds resin to make the surface of an apertured film suitable for contact with the skin of the user of an absorbent article. The resin does not include an opacifier such as titanium dioxide in order to allow the wearer to see the jade particles. The resin for the A layer where the jade is present is simultaneously extruded through a die with a B layer that does not contain any jade particles but has a similar LDPE and LLDPE mixture. The molten blend is then subjected to a pneumatic pressure as it lands on a perforated screen. The pneumatic pressure creates apertures in the PE film and increases its caliper from 25 microns to about 600 microns. The film is then cooled and subjected to a set of intermeshing gears that further thins the surface and exposes the jade particle. The material is then wound on a roll for later application to an absorbent article.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A topsheet comprising a fibrous web or a perforated film, said topsheet having a clear top layer comprising from about 5% to about 50% by weight of jade particles having a mean diameter of from about 10 to about 600 microns and an aspect ratio from about 1 to about 5.

2. The topsheet according to claim 1 wherein the jade particles have a mean diameter of from about 100 to about 300 microns.

3. The topsheet according to claim 2 wherein the jade particles have an aspect ratio from about 1 to about 3.

4. The topsheet according to claim 1 comprising a perforated film comprising thermoplastic material selected from the group consisting of low density polyethylenes, linear low density polyethylenes and mixtures thereof.

5. The topsheet according to claim 4 wherein the jade particles have a mean diameter of from about 100 to about 300 microns and an aspect ratio from about 1 to about 3.

6. The topsheet according to claim 4 wherein the top layer has a textured surface.

7. The topsheet according to claim 6 that has been embossed at a temperature below 70 C after perforating.

8. The topsheet according to claim 7, wherein the embossing pattern is selected from the group consisting of dots, interrupted lines, and continuous lines, and combinations thereof.

9. The topsheet according to claim 1 comprising a nonwoven web.

10. The topsheet according to claim 9 wherein the jade particles have a mean diameter of from about 100 to about 300 microns and an aspect ratio from about 1 to about 3.

11. A method for manufacturing a topsheet for an absorbent article or facial protection mask, comprising the step of forming a clear top layer comprising from about 5% to about 50% by weight of jade particles having a mean diameter of from about 10 to about 600 microns and an aspect ratio from about 1 to about 5 on a fibrous web or film.

12. The method according to claim 11, wherein the film comprises thermoplastic material selected from the group consisting of low density polyethylenes, linear low density polyethylenes and mixtures thereof.

13. The method according to claim 12 wherein the film is coextruded.

14. The method according to claim 12 wherein the surface of the film is textured or perforated.

15. The method according to claim 11 wherein the web or film is printed, stretched, and/or ring-rolled.

16. An absorbent article or facial protection mask comprising a topsheet comprising a fibrous web or a perforated film, said topsheet having a clear top layer comprising from about 5% to about 50% by weight of jade particles having a mean diameter of from about 10 to about 600 microns and an aspect ratio from about 1 to about 5.

17. The article according to claim 16 that it is a diaper, catamenial pad or adult incontinence article.

18. The article or mask according to claim 16 wherein the jade particles have a mean diameter of from about 100 to about 300 microns and an aspect ratio from about 1 to about 3.

19. The article or mask according to claim 16 wherein the topsheet comprises a perforated film comprising thermoplastic material selected from the group consisting of low density polyethylenes, linear low density polyethylenes and mixtures thereof.

20. The article or mask according to claim 16 wherein the topsheet comprises a nonwoven web.
